(19) United States
(12) Patent Application Publication
(10) Pub. No.: US 2023/0210890 A1
(43) Pub. Date: Jul. 6, 2023

(54) COMPOSITIONS AND METHODS OF TREATING COVID-19 WITH HEPARIN OR OTHER NEGATIVELY CHARGED MOLECULES

(71) Applicant: Siemens Healthcare Diagnostics Inc., Tarrytown, NY (US)

(72) Inventor: David Ledden, Elkhart, IN (US)

(73) Assignee: Siemens Healthcare Diagnostics Inc., Tarrytown, NY (US)

(21) Appl. No.: 17/996,086

(22) PCT Filed: Apr. 13, 2021
(86) PCT No.: PCT/US21/26950

§ 371 Date: Oct. 13, 2022

Related U.S. Application Data
(60) Provisional application No. 63/010,956, filed on Apr. 16, 2020.

(51) Int. Cl. A61K 31/727; A61K 31/7012; A61K 45/06

(52) U.S. Cl. CPC A61K 31/727; A61K 31/7012; A61K 45/06

(57) ABSTRACT

Compositions and methods of treating COVID-19 are disclosed. The compositions comprise heparin and/or one or more other negatively charged molecules.

### CROSS REFERENCE TO RELATED APPLICATIONS/INCORPORATION BY REFERENCE

This application claims benefit under 35 USC § 119(e) of U.S. Provisional Application No. 63/010,956, filed Apr. 16, 2020. The entire contents of the above-referenced patent application(s) are hereby expressly incorporated herein by reference.

### STATEMENT REGARDING FEDERALLY SPONSORED RESEARCH OR DEVELOPMENT

Not Applicable.

### BACKGROUND

Newly identified viruses, such as coronaviruses, can be difficult to treat because they are not sufficiently characterized. Severe acute respiratory syndrome coronavirus 2 (SARS-CoV-2) is a newly-emergent coronavirus which causes a severe acute respiratory disease, COVID-19 (coronavirus disease 2019). Clinical features of COVID-19 include fever, dry cough, and fatigue, and the disease can cause respiratory failure resulting in death. The virus spreads readily from person to person primarily through infected secretions, such as saliva and respiratory droplets or aerosols. Evidence supports spread by both symptomatic and asymptomatic individuals.

About 5% of COVID-19 patients experience complications including septic shock, acute respiratory distress syndrome (ARDS), acute cardiac or kidney injury, and disseminated intravascular coagulation (DIC). These complications are thought to be manifestations of the cytokine storm triggered by the host immune response of the virus. In critically ill patients, ARDS was the most common complication in 67% of the patients, with a 28-day mortality of 61.5%. DIC has been widely reported in COVID-19. Pulmonary embolism (PE) in COVID-19 patients has been reported in a few studies. A recent study pointed to a higher incidence of PE with 23% in severe COVID-19 patients.

Many currently available anti-viral agents are being studied to determine their effects on SARS-CoV-2 infection, including ivermectin, remdesivir, chloroquine, and hydroxychloroquine, and new antibody therapies (such as convalescent antibody therapy and monoclonal antibody therapy) are being studied. However, only limited efficacy of approved and experimental anti-viral drugs has been observed.

For example, four repurposed anti-viral drugs (remdesivir, hydroxychloroquine, lopinavir, and interferon beta-1a) were each found to have little or no effect on hospitalized patients with Covid-19 in the WHO Solidarity Trial (WHO Solidarity Trial Consortium; N Engl J Med (published online Dec. 2, 2020) 384:497-511; DOI: 10.1056/NEJMoa2023184). Also, ivermectin has recently been shown to not have a significant effect on the duration of symptoms in adults with mild COVID-19 (López-Medina et al.; JAMA (published online Mar. 04, 2021) doi:10.1001/jama.2021.3071).

In view of the continuing threat to human health, there is an urgent need for new and improved preventive and therapeutic antiviral therapies for SARS-CoV-2 control. It is to such compositions and methods that the present disclosure is directed.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All the compositions, kits, assemblies, and/or methods disclosed herein can be made and executed without undue experimentation in light of the present disclosure. While the compositions, kits, assemblies, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “pharmaceutically-acceptable excipient” refers to any carrier, vehicle, and/or diluent known in the art or otherwise contemplated herein that may improve solubility, deliverability, dispersion, stability, and/or conformational integrity of the compositions disclosed herein.

The terms “patient” and “subject” are used herein interchangeably and will be understood to include human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one infection or condition. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

In addition, an “effective amount” of an active agent of the present disclosure refers to an amount which is effective in controlling, reducing, or inhibiting a condition as described herein, such as (but not limited to) a viral infection and/or the effects associated therewith. The term “controlling” is intended to refer to all processes wherein there may be a slowing, interrupting, arresting, or stopping of the progression of the condition and does not necessarily indicate a total elimination of the symptoms of the condition.

The term “effective amount” is further meant to define an amount resulting in the improvement of any parameters or clinical symptoms characteristic of a condition. The actual dose will vary with the patient's overall condition, the seriousness of the condition or symptoms, and counter indications. As used herein, the term “effective amount” also means the total amount of each active agent (component) of the pharmaceutical composition or method that is sufficient to show a meaningful patient benefit, i.e., reduction of a condition. When applied to an individual active ingredient, administered alone, the term refers to that ingredient alone. When applied to a combination, the term refers to combined amounts of the active agent(s) that results in the therapeutic effect, whether administered in combination, serially or simultaneously.

The term “ameliorate” means a detectable or measurable improvement in a subject's condition or symptom thereof. A detectable or measurable improvement includes a subjective or objective decrease, reduction, inhibition, suppression, limit, or control in the occurrence, frequency, severity, progression, or duration of the condition, or an improvement in a symptom or an underlying cause or a consequence of the condition, or a reversal of the condition. A successful treatment outcome can lead to a “therapeutic effect” or “benefit” of ameliorating, decreasing, reducing, inhibiting, suppressing, limiting, controlling, or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most, or all adverse symptoms, complications, consequences, or underlying causes associated with the condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition (e.g., stabilizing), over a short or long duration of time (e.g., seconds, minutes, hours).

As used herein, the term “concurrent therapy” is used interchangeably with the terms “combination therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concept(s), the treatment of viral infections (such as, but not limited to, COVID-19) using negatively charged molecules is contemplated herein, wherein examples of negatively charged molecules that may be utilized include (but are not limited to) heparin sodium; lithium heparin; sialic acid; a negatively charged polymeric carbohydrate (such as, but not limited to, carboxy methylcellulose); a poly(amino acid) polymer (such as, but not limited to, poly(aspartic acid)(PAsp) or poly(glutamic acid)(PGIu); a poly-nucleic peptide; a poly nucleic acid; an oligo nucleic acid; a peptide nucleic acid (PNA); a polymer containing D-amino acids; or other negative charged polymers; as well as combinations of one or more of any of the above. The negatively charged molecules neutralize binding of virus to healthy cells via inhibition of binding to the highly positive receptor binding domain (RBD) of the SARS-CoV-2 spike protein (S) and thus inhibit infection.

Several avenues are currently being pursued to find treatments for COVID-19, and several therapeutics (such as, but not limited to, virocides, anti-malarial, and anti-inflammatory drugs) and vaccine solutions are being investigated. However, only limited efficacy of approved and experimental anti-viral drugs has been observed.

The present disclosure has shown that certain SARS CoV-2 assays are inhibited by whole blood or plasma samples collected with sodium or lithium heparin anticoagulant. While not wishing to be bound by a particular theory, this phenomenon indicates that heparin is binding to either the Receptor Binding Domain (RBD) of the S1 subunit of the viral spike (S) protein or the Angiotensin Converting Enzyme 2 (ACE2) molecule on the host cell, thus preventing the binding of the virus to the cells and thereby preventing COVID-19 infection. Therefore, administration of heparin to a COVID-19 patient will bind up the virus via binding to the viral S protein or will bind to the ACE2 molecule on the host cell and prevent the virus from being able to interact therewith, thus inhibiting the infection. The use of heparin as an anticoagulant is well known and well used in the art, and many different forms of heparin and heparin salts are widely commercially available for approved for administration to patients. Thus, the ability to administer heparin to a subject is well within the purview of a person having ordinary skill in the art.

Certain non-limiting embodiments of the present disclosure are directed to a pharmaceutical composition for use in the treatment of a viral infection. The pharmaceutical composition comprises at least one negatively charged molecule or a physiologically acceptable salt thereof.

The compositions of the present disclosure may be utilized to treat any viral infections known in the art or otherwise contemplated herein that are responsive to negatively charged molecule(s). For example, but not by way of limitation, the compositions and methods may be utilized to treat a coronavirus infection. Non-limiting examples of coronaviruses that cause infections that can be treated in accordance with the present disclosure include severe acute respiratory syndrome coronavirus (SARS-CoV), severe acute respiratory syndrome coronavirus 2 (SARS-CoV-2, the virus that causes COVID-19), Middle East respiratory syndrome coronavirus (MERS-CoV), human coronavirus 229E (HCoV-229E), human coronavirus OC43 (HCoV-0C43), human coronavirus NL63 (HCoV-NL63), and human coronavirus HKU1 (HCoV-HKU1).

In a particular (but non-limiting) embodiment, the pharmaceutical compositions of the present disclosure are utilized to treat a SARS-CoV-2 infection.

Any negatively charged molecule (or combinations of negatively charged molecules) capable of administration to a patient and capable of functioning as described herein may be utilized in accordance with the present disclosure.

In certain non-limiting embodiments, the negatively charged molecule(s) comprises heparin or a physiologically acceptable salt thereof. Heparin/heparin salts are traditionally used to decrease the clotting ability of the blood and help prevent harmful clots from forming in blood vessels; as such, heparin/heparin salts are well known and widely commercially available. The heparin/heparin salt may be present in any form(s) that allows the heparin to inhibit viral infection as described herein. Non-limiting examples of heparin forms that may be utilized in accordance with the present disclosure include unfractionated heparin, heparinoids, low molecular weight heparin, very low molecular weight heparin, and ultra low molecular weight heparin. Non-limiting examples of physiologically acceptable salts of heparin that may be utilized in accordance with the present disclosure include heparin sodium and lithium heparin.

In addition, the pharmaceutical composition can include a single form of heparin/heparin salt; alternatively, the pharmaceutical composition can include various combinations of different forms of heparin and/or different physiologically acceptable salts of heparin.

In other non-limiting embodiments, the negatively charged molecule(s) present in the pharmaceutical composition comprises sialic acid.

In other non-limiting embodiments, the negatively charged molecule(s) present in the pharmaceutical composition comprises at least one negatively charged polymer. Any negatively charged polymers capable of administration to a patient and capable of inhibiting viral infection as described herein may be utilized in accordance with the present disclosure. Non-limiting examples of negatively charged polymers that may be utilized in accordance with the present disclosure a negatively charged polymeric carbohydrate (such as, but not limited to, carboxy methylcellulose); a poly(amino acid) polymer (such as, but not limited to, poly(aspartic acid) (PAsp) or poly(glutamic acid)(PGlu)); a poly-nucleic peptide; a poly nucleic acid; an oligo nucleic acid; a peptide nucleic acid (PNA); a polymer containing D-amino acids; and combinations thereof.

In certain non-limiting embodiments, the pharmaceutical composition comprises two or more negatively charged polymers.

In certain non-limiting embodiments, the pharmaceutical composition comprises at least one form of heparin/heparin salt and/or sialic acid as well as at least one negatively charged polymer.

In at least certain non-limiting embodiments, the negatively charged molecule(s) of the present disclosure may be combined with one or more secondary compounds, such as (but not limited to) a pharmaceutically acceptable component (e.g., a carrier, vehicle, excipient, and/or diluent), to form the pharmaceutical composition. For example (but not by way of limitation), the pharmaceutical composition may contain, in addition to the negatively charged molecule(s), one or more of a carrier, a diluent, a filler, a salt, a buffer, a stabilizer, a solubilizer, and other materials well known in the art, as well as any combination thereof. Suitable carriers, vehicles, and other components for pharmaceutical formulations are described, for example, in Remington: The Science and Practice of Pharmacy, 23rd ed (2020).

Where used herein, the term “secondary compound” refers to any compound used in combination (e.g., in a composition or formulation) with the negatively charged molecule(s) of the present disclosure, for example, for aiding in delivery of the negatively charged molecule(s) to the subject to be treated. The term “pharmaceutically acceptable” means a non-toxic material that does not interfere with the effectiveness of the biological activity of the negatively charged molecule(s). The characteristics of the carrier will depend on the route of administration.

For example, but not by way of limitation, the negatively charged molecule(s) may be dissolved in a secondary compound such as a physiologically acceptable pharmaceutical carrier, vehicle, excipient, or diluent and administered as either a solution or a suspension. Non-limiting examples of suitable pharmaceutically acceptable carriers include water, saline, dextrose solutions, fructose solutions, ethanol, or oils of animal, vegetative, or synthetic origin, or any combination thereof. A sterile diluent, which may contain materials generally recognized for approximating physiological conditions and/or as required by governmental regulations, may be employed as the pharmaceutically acceptable carrier. In this respect, the sterile diluent may contain a buffering agent to obtain a physiologically acceptable pH, such as (but not limited to) sodium chloride, saline, phosphate-buffered saline, and/or other substances which are physiologically acceptable and/or safe for use.

The pharmaceutical compositions may also contain one or more additional components in addition to the negatively charged molecule(s) (and pharmaceutically acceptable carrier(s)/excipient(s), if present). Examples of additional secondary compounds that may be present include, but are not limited to, diluents, fillers, salts, buffers, preservatives, stabilizers, solubilizers, and other materials well known in the art.

The negatively charged molecule(s) may be present in the pharmaceutical compositions at any concentration that allows the pharmaceutical composition to function in accordance with the present disclosure; for example, but not by way of limitation, the negatively charged molecule(s) may be present at a concentration of about 0.0001 wt %, about 0.005 wt %, about 0.001 wt %, about 0.005 wt %, about 0.01 wt %, about 0.05 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, and about 2 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, or higher. In addition, the negatively charged molecule(s) may be present at a concentration that falls within a range formed of two of the above values (i.e., a range of from about 0.001 wt % to about 75 wt %, a range of from about 0.05 wt % to about 35 wt %, etc.); also, the negatively charged molecule(s) may be present at a concentration that falls within a range of two values, each of which falls between two values listed above (i.e., a range of from about 0.03 wt % to about 22 wt %; a range of from about 0.08 wt % to about 63 wt %; etc.).

In addition, in certain non-limiting embodiments, the negatively charged molecule(s) may each be present in the pharmaceutical composition at a specific molar concentration. Non-limiting examples of molar concentrations that may be utilized in accordance with the present disclosure include about 0.0001 M, about 0.0005 M, about 0.001 M, about 0.005 M, about 0.01 M, about 0.05 M, about 0.1 M, about 0.2 M, about 0.3 M, about 0.4 M, about 0.5 M, about 0.6 M, about 0.7 M, about 0.8 M, about 0.9 M, about 1 M, about 2 M, about 3 M, about 4 M, about 5 M, or higher. In addition, the negatively charged molecule(s) may be present at a molar concentration that falls within a range formed of two of the above values (i.e., a range of from about 0.0001 M to about 1 M, a range of from about 0.001 M to about 0.1 M, etc.); also, the negatively charged molecule(s) may be present at a concentration that falls within a range of two values, each of which falls between two values listed above (i.e., a range of from about 0.007 M to about 0.86 M, etc.).

When the negatively charged molecule(s) present in the pharmaceutical composition include heparin or a salt thereof, the level of heparin/heparin salt present in the pharmaceutical composition may be based upon units of heparin activity (i.e., anticoagulant activity) per milligram. For example (but not by way of limitation), heparin/heparin salt may be present in the pharmaceutical composition in a range of from about 1 USP unit per mL to about 50,000 USP units per mL. Particular (but non-limiting) examples of heparin/heparin salt concentrations include about 1 USP unit per mL, about 2 USP units per mL, about 3 USP units per mL, about 4 USP units per mL, about 5 USP units per mL, about 6 USP units per mL, about 7 USP units per mL, about 8 USP units per mL, about 9 USP units per mL, about 10 USP units per mL, about 15 USP units per mL, about 20 USP units per mL, about 25 USP units per mL, about 30 USP units per mL, about 35 USP units per mL, about 40 USP units per mL, about 45 USP units per mL, about 50 USP units per mL, about 60 USP units per mL, about 70 USP units per mL, about 80 USP units per mL, about 90 USP units per mL, about 100 USP units per mL, about 125 USP units per mL, about 150 USP units per mL, about 175 USP units per mL, about 200 USP units per mL, about 250 USP units per mL, about 300 USP units per mL, about 350 USP units per mL, about 400 USP units per mL, about 450 USP units per mL, about 500 USP units per mL, about 550 USP units per mL, about 600 USP units per mL, about 650 USP units per mL, about 700 USP units per mL, about 750 USP units per mL, about 800 USP units per mL, about 850 USP units per mL, about 900 USP units per mL, about 1000 USP units per mL, about 2000 USP units per mL, about 3000 USP units per mL, about 4000 USP units per mL, about 5000 USP units per mL, about 6000 USP units per mL, about 7000 USP units per mL, about 8000 USP units per mL, about 9000 USP units per mL, about 10000 USP units per mL, about 11000 USP units per mL, about 12000 USP units per mL, about 13000 USP units per mL, about 14000 USP units per mL, about 15000 USP units per mL, about 16000 USP units per mL, about 17000 USP units per mL, about 18000 USP units per mL, about 19000 USP units per mL, about 20000 USP units per mL, about 25000 USP units per mL, about 30000 USP units per mL, about 35000 USP units per mL, about 40000 USP units per mL, about 45000 USP units per mL, about 50000 USP units per mL, or higher, or any range formed of two of the above values (a range of from about 1 USP unit per mL to about 100 USP units per mL, a range of from about 1,000 USP units per mL to about 20,000 USP units per mL, etc.), or a range formed of two values that each fall between two of the above values (i.e., a range of from about 625 USP units per mL to about 32,000 USP units per mL, etc.).

Certain non-limiting embodiments of the present disclosure are directed to a method for treating a viral infection in a subject in need thereof. In the method, any of the pharmaceutical compositions disclosed or otherwise contemplated herein is administered to a subject in need.

Any viral infections known in the art or otherwise contemplated herein that are responsive to negatively charged molecule(s) may be treated with the methods of the present disclosure. For example, but not by way of limitation, the methods of the present disclosure may be utilized to treat a coronavirus infection. Non-limiting examples of coronaviruses that cause infections that can be treated in accordance with the present disclosure include severe acute respiratory syndrome coronavirus (SARS-CoV), severe acute respiratory syndrome coronavirus 2 (SARS-CoV-2, the virus that causes COVID-19), Middle East respiratory syndrome coronavirus (MERS-CoV), human coronavirus 229E (HCoV-229E), human coronavirus OC43 (HCoV-0C43), human coronavirus NL63 (HCoV-NL63), and human coronavirus HKU1 (HCoV-HKU1).

The amount of the negatively charged molecule(s) present in the pharmaceutical composition that is effective in the treatment described herein can be determined by the attending diagnostician, as one of ordinary skill in the art, by the use of conventional techniques and by observing results obtained under analogous circumstances. In determining the therapeutically effective dose, a number of factors may be considered by the attending diagnostician, including, but not limited to: the species of the subject; its size, age, and general health; the specific diseases, infections, and/or other conditions involved; the degree, involvement, and/or severity of the diseases, infections, and/or conditions; the response of the individual subject; the particular negatively charged molecule(s) administered; the mode of administration; the dose regimen selected; the use of concomitant medication; and other relevant circumstances. A therapeutically effective amount of the negatively charged molecule(s) of the present disclosure also refers to an amount of the negatively charged molecule(s) which is effective in controlling, reducing, or ameliorating the condition/infection to be treated or may refer to the amount of the negatively charged molecule(s) required to achieve a prophylactic effect for the purpose of preventing, controlling, reducing, or ameliorating the condition/infection to be treated.

For example, but not by way of limitation, the therapeutically effective amount of pharmaceutical composition will generally contain sufficient negatively charged molecule(s) to deliver in a range of from about 0.01 μg/kg to about 10 mg/kg (weight of negatively charged molecule(s)/body weight of patient). For example, but not by way of limitation, the composition will deliver about 0.1 μg/kg to about 5 mg/kg, and more particularly about 1 μg/kg to about 1 mg/kg of the negatively charged molecule(s).

Exemplary, non-limiting ranges for a therapeutically or prophylactically effective amount of the negatively charged molecule(s) include but are not limited to a range of from about 0.001 mg/kg of the subject's body weight to about 100 mg/kg of the subject's body weight, a range of from about 0.01 mg/kg to about 100 mg/kg, a range of from about 0.1 mg/kg to about 50 mg/kg, a range of from about 0.1 mg/kg to about 40 mg/kg, a range of from about 1 mg/kg to about 30 mg/kg, a range of from about 1 mg/kg to about 20 mg/kg, a range of from about 2 mg/kg to about 30 mg/kg, a range of from about 2 mg/kg to about 20 mg/kg, a range of from about 2 mg/kg to about 15 mg/kg, a range of from about 2 mg/kg to about 12 mg/kg, a range of from about 2 mg/kg to about 10 mg/kg, a range of from about 3 mg/kg to about 30 mg/kg, a range of from about 3 mg/kg to about 20 mg/kg, a range of from about 3 mg/kg to about 15 mg/kg, a range of from about 3 mg/kg to about 12 mg/kg, a range of from about 3 mg/kg to about 10 mg/kg, a range of from about 5 mg to about 1500 mg, and the like, as a fixed dosage.

The pharmaceutical composition is formulated to contain an effective amount of the negatively charged molecule(s), wherein the amount depends on the animal to be treated and the condition to be treated. In certain embodiments, the negatively charged molecule(s) is administered at a dose ranging from about 0.001 mg to about 10 g, a dose ranging from about 0.01 mg to about 10 g, a dose ranging from about 0.1 mg to about 10 g, a dose ranging from about 1 mg to about 10 g, a dose ranging from about 1 mg to about 9 g, a dose ranging from about 1 mg to about 8 g, a dose ranging from about 1 mg to about 7 g, a dose ranging from about 1 mg to about 6 g, a dose ranging from about 1 mg to about 5 g, a dose ranging from about 10 mg to about 10 g, a dose ranging from about 50 mg to about 5 g, a dose ranging from about 50 mg to about 5 g, a dose ranging from about 50 mg to about 2 g, a dose ranging from about 0.05 μg to about 1.5 mg, a dose ranging from about 10 μg to about 1 mg, a dose ranging from about 30 μg to about 500 μg, a dose ranging from about 0.1 μg to about 200 mg, a dose ranging from about 0.1 μg to about 5 μg, a dose ranging from about 5 μg to about 10 μg, a dose ranging from about 10 μg to about 25 μg, a dose ranging from about 25 μg to about 50 μg, a dose ranging from about 50 μg to about 100 μg, a dose ranging from about 100 μg to about 500 μg, a dose ranging from about 500 μg to about 1 mg, or a dose ranging from about 1 mg to about 2 mg. The specific dosage level for any particular subject depends upon a variety of factors including the activity of the specific peptide, the age, body weight, general health, sex, diet, time of administration, route of administration, and rate of excretion, drug combination, and the severity of the particular disease undergoing therapy.

The dosage of an administered pharmaceutical composition for the subject will vary depending upon such factors as the patient's age, weight, height, sex, general medical condition, and previous medical history. In certain non-limiting embodiments, the recipient is provided with a dosage of the negatively charged molecule(s) that is in the range of from about 1 mg to about 1000 mg as a single infusion or single or multiple injections, although a lower or higher dosage also may be administered. The dosage may be in the range of from about 25 mg to about 100 mg of the negatively charged molecule(s) per square meter (m²) of body surface area for a typical adult, although a lower or higher dosage also may be administered. Examples of dosages that may be administered to a human subject further include, for example, in a range of from about 1 mg to about 500 mg, a range of from about 1 mg to about 70 mg, or a range of from about 1 mg to about 20 mg, although higher or lower doses may be used. Dosages may be repeated as needed, for example (but not by way of limitation), once every 10 minutes, once every 30 minutes, once every hour, once every two hours, once every three hours, once every four hours, once every five hours once every six hours, once every eight hours, once every 12 hours, once a day, once per week, etc. It may also be given less frequently, such as every other week for several months, or more frequently, such as twice weekly, or by continuous infusion.

When the pharmaceutical composition contains heparin/heparin salt, the pharmaceutical composition may be administered to the subject in an amount that provides a heparin/heparin salt dose per administration of about 1 USP unit, about 5 USP units, about 10 USP units, about 20 USP units, about 30 USP units, about 40 USP units, about 50 USP units, about 60 USP units, about 70 USP units, about 80 USP units, about 90 USP units, about 100 USP units, about 200 USP units, about 300 USP units, about 400 USP units, about 500 USP units, about 600 USP units, about 700 USP units, about 800 USP units, about 900 USP units, about 1000 USP units, about 5000 USP units, about 10000 USP units, about 20000 USP units, about 30000 USP units, about 40000 USP units, about 50000 USP units, about 60000 USP units, about 70000 USP units, about 80000 USP units, about 90000 USP units, about 100000 USP units, about 110000 USP units, about 120000 USP units, or higher, or any range formed of two of the above values (a range of from about 1 USP unit to about 10,000 USP units, a range of from about 100 USP units to about 120,000 USP units, etc.), or a range formed of two values that each fall between two of the above values (i.e., a range of from about 625 USP units to about 32,000 USP units, etc.). Dosages may be repeated as needed, for example (but not by way of limitation), once every 10 minutes, once every 30 minutes, once every hour, once every two hours, once every three hours, once every four hours, once every five hours once every six hours, once every eight hours, once every 12 hours, once a day, once per week, etc. It may also be given less frequently, such as every other week for several months, or more frequently, such as twice weekly, or by continuous infusion.

The pharmaceutical compositions of the present disclosure may be administered via one or more routes of administration using one or more of a variety of methods known in the art. As will be appreciated by the skilled artisan, the route and/or mode of administration will vary depending upon the desired results. Selected routes of administration include (but are not limited to) intravenous, intramuscular, intradermal, intraperitoneal, subcutaneous, spinal, or other parenteral routes of administration, for example by injection or infusion. The negatively charged molecule(s) can be delivered alone or as pharmaceutical compositions by any means known in the art, e.g., systemically, regionally, or locally; by intra-arterial, intrathecal (IT), intravenous (IV), parenteral, intra-pleural cavity, or local administration, as subcutaneous, intra-tracheal (e.g., by aerosol), or transmucosal (e.g., buccal, bladder, vaginal, uterine, rectal, nasal mucosa). Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal injection and infusion. Alternatively, compositions can be administered via a non-parenteral route, such as a topical, epidermal, or mucosal route of administration, for example, intranasally, orally, vaginally, rectally, sublingually, or topically. In one embodiment, the composition is administered by infusion. In another embodiment, the composition is administered subcutaneously. In another embodiment, the composition is administered orally. In another embodiment, the composition is administered to the ear canal. In another embodiment, the composition is administered transdermally. In another embodiment, the composition is administered to the lungs with no penetration, partial penetration, or complete penetration of the lung tissues.

Compositions for inhalation or insufflation include solutions and suspensions in pharmaceutically acceptable, aqueous, or organic solvents, or mixtures thereof, and powders. The liquid or solid compositions may contain suitable pharmaceutically acceptable excipients as described supra.

The compositions can be administered by the oral or nasal respiratory route for local or systemic effect. Compositions in pharmaceutically acceptable solvents may be nebulized by use of inert gases. Nebulized solutions may be inhaled directly from the nebulizing device, or the nebulizing device may be attached to a face mask tent or intermittent positive pressure breathing machine. Solution, suspension, or powder compositions may also be administered orally or nasally from devices which deliver the formulation in an appropriate manner.

As noted, the negatively charged molecule(s) can be combined with a pharmaceutically acceptable carrier (excipient) or vehicle to form a pharmacological composition. Pharmaceutically acceptable carriers can contain a physiologically acceptable compound that acts to stabilize, increase, or decrease the absorption or clearance rates of the pharmaceutical compositions. Physiologically acceptable carriers and vehicles can include, for example (but not by way of limitation), carbohydrates, such as glucose, sucrose, or dextrans; antioxidants, such as ascorbic acid or glutathione; chelating agents; low molecular weight proteins; detergents; liposomal carriers; or excipients or other stabilizers and/or buffers. Other non-limiting examples of physiologically acceptable compounds, carriers, and vehicles include wetting agents, emulsifying agents, dispersing agents, or preservatives.

When the pharmaceutical composition is to be used as an injectable material, it can be formulated into a conventional injectable carrier. Non-limiting examples of suitable carriers include biocompatible and pharmaceutically acceptable phosphate buffered saline solutions, which are particularly isotonic.

In certain non-limiting embodiments, the negatively charged molecule(s) of the injectable material is provided as a lyophilized product that is reconstituted prior to injection. For reconstitution of a lyophilized product in accordance with the present disclosure, one may employ a sterile diluent, which may contain materials generally recognized for approximating physiological conditions and/or as required by governmental regulation. In this respect, the sterile diluent may contain a buffering agent to obtain a physiologically acceptable pH, such as (but not limited to) sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition may also be in the form of an aqueous solution containing many of the same substances as described above for the reconstitution of a lyophilized product.

Practice of the method of the present disclosure may include administering to a subject a therapeutically effective amount of the pharmaceutical composition (containing the negatively charged molecule(s) in any suitable systemic and/or local formulation), in an amount effective to deliver the desired dosage. The dosage can be administered, for example, but not by way of limitation, continuously or intermittently. In addition, the dosage can be administered on a one-time basis or administered at multiple times (for example, but not by way of limitation, from one to five times per day, or once or twice per week). The pharmaceutical composition may be administered either alone or in combination with other therapies, in accordance with the inventive concepts disclosed herein.

The pharmaceutical compositions of the present disclosure can be administered in a single dose treatment or in multiple dose treatments on a schedule and over a time period appropriate to the age, weight, and condition of the subject, the particular composition used, and the route of administration. In one embodiment, a single dose of the composition according to the disclosure is administered. In other embodiments, multiple doses are administered. The frequency of administration can vary depending on any of a variety of factors, e.g., severity of the symptoms, whether the composition is used for prophylactic or curative purposes, etc. For example, in certain non-limiting embodiments, the composition is administered once per day, twice per day, three times per day, four times per day, five times per day, six times per day, seven times per day, eight times per day, nine times per day, 10 times per day, 12 times per day, or more frequently, or the composition may be administered every other day, once per week, twice per week, three times per week, four times per week, five times per week, six times per week, or seven times per week. The duration of treatment, e.g., the period of time over which the composition is administered, can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, or more.

In certain particular (but non-limiting) embodiments of the present disclosure, the method is a method for treating a severe acute respiratory syndrome coronavirus 2 (SARS-CoV-2) infection in a subject in need thereof, and the pharmaceutical composition administered to the subject comprises heparin or a physiologically acceptable salt thereof. The heparin may be administered alone or in combination with other negatively charged molecule(s). For example (but not by way of limitation), the pharmaceutical composition may further comprise at least one negatively charged polymer. Alternatively, the method may further comprise the step of administering a second pharmaceutical composition to the subject, wherein the second pharmaceutical composition comprises at least one negatively charged polymer. Any of the negatively charged polymers or physiologically acceptable salts thereof disclosed or otherwise contemplated herein may be utilized. When the negatively charged polymer(s) is present in a second pharmaceutical composition, the second pharmaceutical composition may be administered simultaneously or wholly or partially sequentially with the pharmaceutical composition comprising heparin or a physiologically acceptable salt thereof.

In other particular (but non-limiting) embodiments of the present disclosure, the method is a method for treating a severe acute respiratory syndrome coronavirus 2 (SARS-CoV-2) infection in a subject in need thereof, and the pharmaceutical composition administered to the subject comprises a negatively charged polymer or a physiologically acceptable salt thereof. Any of the negatively charged polymers or physiologically acceptable salts thereof disclosed or otherwise contemplated herein may be utilized.

In certain particular (but non-limiting) embodiments, the pharmaceutical composition is systemically administered to the subject.

In certain particular (but non-limiting) embodiments, the pharmaceutical composition is administered intravenously.

In certain particular (but non-limiting) embodiments, the pharmaceutical composition is administered subcutaneously.

In certain particular (but non-limiting) embodiments, the pharmaceutical composition is administered via an aerosol inhaler.

In certain particular (but non-limiting) embodiments, the pharmaceutical composition is administered via a nebulizer.

In certain particular (but non-limiting) embodiments, the pharmaceutical composition is administered continuously to the subject.

In certain particular (but non-limiting) embodiments, the pharmaceutical composition is administered intermittently to the subject.

In certain particular (but non-limiting) embodiments, two or more doses of the pharmaceutical composition are administered to the subject over a period of time.

In certain particular (but non-limiting) embodiments, the pharmaceutical compositions of the present disclosure are administered concurrently with one or more additional agents (such as, but not limited to, one or more of an anti-viral drug, an anti-malarial drug, an anti-inflammatory drug, or combinations thereof). When concurrent therapy is utilized, the two or more compositions may be administered simultaneously or wholly or partially sequentially.

While the above disclosures describe the inventive concept(s) in conjunction with the specific experimentation, results, and language set forth herein, it is evident that many alternatives, modifications, and variations will be apparent to those skilled in the art. Accordingly, it is intended to embrace all such alternatives, modifications, and variations that fall within the spirit and broad scope of the present disclosure.

1. A method for treating a viral infection in a subject in need thereof, comprising:
administering to the subject a pharmaceutical composition comprising at least one negatively charged molecule or a physiologically acceptable salt thereof.

2. The method of claim 1, wherein the viral infection is caused by severe acute respiratory syndrome coronavirus 2 (SARS-CoV-2).

3. The method of claim 1, wherein the at least one negatively charged molecule comprises heparin.

4. The method of claim 3, wherein heparin is in the form of heparin sodium.

5. The method of claim 3, wherein the pharmaceutical composition has a concentration of heparin in a range of from about 1 USP unit per mL to about 40,000 USP units per mL.

6. The method of claim 5, wherein an amount of pharmaceutical composition administered to the subject provides a heparin dosage in a range of from about 100 USP units to about 120,000 USP units.

7. The method of claim 1, wherein the at least one negatively charged molecule comprises sialic acid.

8. The method of claim 1, wherein the at least one negatively charged molecule comprises a negatively charged polymer selected from the group consisting of a negatively charged polymeric carbohydrate, a poly(amino acid) polymer, a poly-nucleic peptide, a poly nucleic acid, an oligo nucleic acid, a peptide nucleic acid (PNA), a polymer containing D-amino acids, and combinations thereof.

9. The method of claim 8, wherein the negatively charged polymer is selected from the group consisting of carboxy methylcellulose, poly(aspartic acid) (PAsp), poly(glutamic acid) (PGlu), and combinations thereof.

10. The method of claim 1, wherein the pharmaceutical composition is administered systemically, intravenously, or subcutaneously to the subject.

11. The method of claim 1, wherein the pharmaceutical composition is administered via an aerosol inhaler or a nebulizer.

12. The method of claim 1, wherein the pharmaceutical composition is administered continuously to the subject.

13. The method of claim 1, wherein the pharmaceutical composition is administered intermittently to the subject.

14. A method for treating a severe acute respiratory syndrome coronavirus 2 (SARS-CoV-2) infection in a subject in need thereof, comprising:
administering to the subject a pharmaceutical composition comprising heparin or a physiologically acceptable salt thereof.

15. The method of claim 14, wherein heparin is in the form of heparin sodium.

16. The method of claim 14, wherein the pharmaceutical composition further comprises a negatively charged molecule selected from the group consisting of sialic acid, a negatively charged polymeric carbohydrate, a poly(amino acid) polymer, a poly-nucleic peptide, a poly nucleic acid, an oligo nucleic acid, a peptide nucleic acid (PNA), a polymer containing D-amino acids, and combinations thereof.

17. The method of claim 16, wherein the negatively charged molecule is selected from the group consisting of carboxy methylcellulose, poly(aspartic acid) (PAsp), poly(glutamic acid) (PGlu), and combinations thereof.

18. The method of claim 14, further comprising the step of administering a second pharmaceutical composition to the subject, wherein the second pharmaceutical composition comprises a negatively charged molecule selected from the group consisting of sialic acid, a negatively charged polymeric carbohydrate, a poly(amino acid) polymer, a poly-nucleic peptide, a poly nucleic acid, an oligo nucleic acid, a peptide nucleic acid (PNA), a polymer containing D-amino acids, and combinations thereof.

19. The method of claim 18, wherein the negatively charged molecule is selected from the group consisting of carboxy methylcellulose, poly(aspartic acid) (PAsp), poly(glutamic acid) (PGlu), and combinations thereof.

20. The method of claim 18, wherein the second pharmaceutical composition is administered wholly or partially sequentially with the pharmaceutical composition comprising heparin or a physiologically acceptable salt thereof.

21. A method for treating a severe acute respiratory syndrome coronavirus 2 (SARS-CoV-2) infection in a subject in need thereof, comprising:
administering to the subject a pharmaceutical composition comprising a negatively charged molecule or a physiologically acceptable salt thereof, wherein the negatively charged molecule is selected from the group consisting of sialic acid, a negatively charged polymeric carbohydrate, a poly(amino acid) polymer, a poly-nucleic peptide, a poly nucleic acid, an oligo nucleic acid, a peptide nucleic acid (PNA), a polymer containing D-amino acids, and combinations thereof.

22. The method of claim 21, wherein the negatively charged molecule is selected from the group consisting of carboxy methylcellulose, poly(aspartic acid) (PAsp), poly(glutamic acid) (PGlu), and combinations thereof.

23. The method of claim 1, wherein the at least one negatively charged molecule comprises heparin.

24. The method of claim 23, wherein heparin is in the form of heparin sodium.

25. The method of claim 23, wherein the pharmaceutical composition has a concentration of heparin in a range of from about 1 USP unit per mL to about 40,000 USP units per mL.

26. The method of claim 25, wherein an amount of pharmaceutical composition administered to the subject provides a heparin dosage in a range of from about 100 USP units to about 120,000 USP units.

27. The method of claim 1, wherein the at least one negatively charged molecule comprises sialic acid.

28. The method of claim 1, wherein the at least one negatively charged molecule comprises a negatively charged polymer selected from the group consisting of a negatively charged polymeric carbohydrate, a poly(amino acid) polymer, a poly-nucleic peptide, a poly nucleic acid, an oligo nucleic acid, a peptide nucleic acid (PNA), a polymer containing D-amino acids, and combinations thereof.

29. The method of claim 28, wherein the negatively charged polymer is selected from the group consisting of carboxy methylcellulose, poly(aspartic acid) (PAsp), poly(glutamic acid) (PGlu), and combinations thereof.

30. The method of claim 1, wherein the pharmaceutical composition is administered systemically, intravenously, or subcutaneously to the subject.

31. The method of claim 1, wherein the pharmaceutical composition is administered via an aerosol inhaler or a nebulizer.

32. The method of claim 1, wherein the pharmaceutical composition is administered continuously to the subject.

33. The method of claim 1, wherein the pharmaceutical composition is administered intermittently to the subject.

34. The method of claim 14, wherein the pharmaceutical composition further comprises a negatively charged molecule selected from the group consisting of sialic acid, a negatively charged polymeric carbohydrate, a poly(amino acid) polymer, a poly-nucleic peptide, a poly nucleic acid, an oligo nucleic acid, a peptide nucleic acid (PNA), a polymer containing D-amino acids, and combinations thereof.

35. The method of claim 34, wherein the negatively charged molecule is selected from the group consisting of carboxy methylcellulose, poly(aspartic acid) (PAsp), poly(glutamic acid) (PGlu), and combinations thereof.

36. The method of claim 14, further comprising the step of administering a second pharmaceutical composition to the subject, wherein the second pharmaceutical composition comprises a negatively charged molecule selected from the group consisting of sialic acid, a negatively charged polymeric carbohydrate, a poly(amino acid) polymer, a poly-nucleic peptide, a poly nucleic acid, an oligo nucleic acid, a peptide nucleic acid (PNA), a polymer containing D-amino acids, and combinations thereof.

37. The method of claim 36, wherein the negatively charged molecule is selected from the group consisting of carboxy methylcellulose, poly(aspartic acid) (PAsp), poly(glutamic acid) (PGlu), and combinations thereof.

38. The method of claim 18, wherein the second pharmaceutical composition is administered wholly or partially sequentially with the pharmaceutical composition comprising heparin or a physiologically acceptable salt thereof.
